(19) United States
(12) Patent Application Publication
(10) Pub. No.: US 2015/0133842 A1
(43) Pub. Date: May 14, 2015

(54) TENSIONING SYSTEM FOR DORSAL ORTHOPEDIC SUPPORTS FOR THE VERTEBRAL COLUMN

(71) Applicant: F.G.P. S.r.I., Villafranca, VR (IT)

(72) Inventors: Moreno Ferrigolo, Villafranca di Verona (IT); Alberto Turrini, Villafranca di Verona (IT)

(73) Assignee: F.G.P. S.r.I., Villafranca, VR (IT)

(21) Appl. No.: 14/395,286

(22) PCT Filed: Apr. 11, 2013
(86) PCT No.: PCT/EP2013/057640

§ 371 Date: Oct. 17, 2014

(30) Foreign Application Priority Data
Apr. 20, 2012 (IT) VR2012A000076

(51) Int. Cl. A61F 5/02

(52) U.S. Cl. CPC A61F 5/026
USPC 602/19

(57) ABSTRACT

A system for tensioning straps applied to dorsal orthopedic supports used to support and restrain the vertebral column, said orthoses consisting of a semirigid bar (10) wherein the lower part is coupled to a lower strap (12) which is fixed anteriorly to an abdominal plate (13) that encircles the waist, and the middle part is coupled to intermediate straps (14) that terminate at loops (15) positioned at the sides of the body, there also being additional vertical straps (16) that start from the abdominal plate (13), pass through the loops (15) and terminate above the shoulder where the ends are fixed to loops (17), whereby said loops (17) fitted to the ends of the upper straps (16) are coupled to respective cables (26) which can be tensioned by a mobile slide (21) in a sliding housing (22) by means of tie rods (18) coupled to the orthosis (10) and activated by the patient by means of relative handles (24) positioned at their lower end.

### TECHNICAL FIELD

This invention concerns a particular system for tensioning of the straps fitted on dorsal orthopedic supports, that is to say on dorsal orthoses which can be used to support and block the spinal column of subjects with articular problems of the back or suffering from osteoporosis.

More specifically, this invention refers to a tensioning system for spinal supports that, with respect to known solutions, presents the advantage of allowing correct support of the back against the rod of the orthosis positioned against the patient's spinal column and greater straightening of the trunk.

This invention can be applied in the medical and orthopedic industry and particularly in the production sector of orthopedic jackets in general but also of prostheses and braces mainly used in conservative, post-traumatic, rehabilitation and post-operative therapy.

### BACKGROUND ART

It is known that with some diseases or some orthopedic type disorders of the spinal column or the trunk in persons subject for example to osteoporosis or other degenerative inflammatory disorders, or following injury, it is necessary to wear particular jacket-type or back braces or orthoses which guarantee a certain degree of support for the patient, absorbing the most intense stresses that the trunk is submitted to.

Particularly useful in all types of backache in less acute phases of osteoporosis, by activating the muscles in the back, the brace favours straightening of the trunk, reducing the kyphosis caused by osteoporosis.

Various types of jackets, braces or orthoses designed to support and contain the trunk are currently known and available. These are generally structures which rest against the spinal column and which mainly consist of a plate which stiffens the vertebral column, and thus has the same shape, and which is attached to the trunk and held in place by means of straps.

These devices consist, in fact, of a rigid elongated frame, made from metal and shaped to adhere to the vertebral column, the frame being fixed to the user's trunk by fastening means which are usually the strap or jacket type.

If strap fastening means are used, these consist of harnesses with ends attached to the rigid frame and which wrap around the trunk and are secured in place by appropriate adjustable type means of restraint.

These straps are connected to the central body of the brace positioned in correspondence with the vertebral column and are in contact with various anatomical parts of the body.

In particular, it is possible to distinguish between two types of straps that are attached to a back brace and which allow it to be fixed to the trunk:
a first upper strap, which starts from the upper end of the back brace and which, passing below the shoulder-humerus joint, has an elastic element which is positioned more or less at the mid point of the brace and which terminates on the front support plate that must be positioned in the patient's abdominal area;
a second strap which starts from the lower part of the back brace, passing over the patient's sides, and terminates on the aforesaid plate.

This system of straps must be adjusted in length in order to guarantee the correct adherence of the brace to the patient's vertebral column and the correct thrust system that acts on the anatomical parts involved.

The technical problem encountered with these solutions, the general use of which is still valid, consists of the fact that there are cases, such as for example in the presence of hyperkyphotic patients, in which the upper end of the brace tends to be shifted with respect to the line of the spinal column and, thus, the support is not effective.

In other words, in these specific cases, that is in the presence of hyperkyphosis, the brace combined with traditional straps does not represent a valid and complete aid for the correction of the specific disease, and is in fact of little use.

Document WO 2007/043079 A1 discloses an orthopedic support which can be used to support and block the spinal column of subjects with articular back problems or suffering from osteoporosis, comprising at least one elongated rigid splint with the anatomical shape of the spinal column. The splint is contained inside a pocket made from soft cloth or velvet. The pocket presents attachment areas or tabs for straps for fastening the orthesis to the trunk of the wearer and divided into three groups consisting of upper straps, which wrap around the shoulders, middle straps and lower or lumbar straps shich respectively wrap around the sides of the body and the waist close to the buttocks, the middle and lower straps meeting at the front of the trunk in an abdominal connection plate.

Document US 2011/0152737 A1 discloses a posture correction brace for a person, having a tightening mechanism that allows a user to pull a cord to pull down on the back of a pair of shoulder straps, thereby pulling the shoulders back to correct the person's posture. The front ends of the shoulder straps are typically also attached to the back of the wearer, to help the posture correction brace pull the shoulders back as the shoulders straps tighten.

### DESCRIPTION OF THE INVENTION

This invention proposes to provide a tensioning system for dorsal supports for the correction and/or support of the vertebral column, which can eliminate or at least reduce the problems described above.

In particular, the tensioning system for dorsal supports according to the invention proposes to overcome the problems caused by the lack of adherence of the brace to the vertebral column in the case of patients with hyperkyphosis problems, allowing in particular the upper part of the brace to adhere to the curvature of the upper arch of the vertebral column.

The invention also proposes to provide a tensioning system for dorsal supports that can be easily produced in order to be economically advantageous as well as extremely efficient from the point of view of safety and stability.

This is achieved by means of a tensioning system for dorsal supports with the characteristics described in the main claim.

The dependent claims describe advantageous embodiments of the invention.

The proposed aims are achieved, according to the invention, by a tensioning system for dorsal supports made by using special tie rods designed to act on the straps which are arranged to form a sort of harness starting from the orthosis itself protected by an appropriately shaped covering.

The tensioning system of the dorsal support according to the invention therefore foresees the use of tie rods which, appropriately fitted, act on the system of straps described above.

In particular, the system acts on the “upper strap” which is no longer connected rigidly to the brace but to a loop which is part of the system in question. This loop is connected, by means of a cable, to a block-pulley system which is housed in a slot appropriately positioned in the upper part of the orthosis.

The use of the tensioning system according to the invention foresees that, by operating the handles positioned at the height of the hips and pulling the cables attached to the handles, thanks to the geometry of the cable passage, the patient causes the downward movement of the block.

### DESCRIPTION OF THE DRAWINGS

Other features and advantages of the invention will become clear on reading the description given below of one embodiment, provided as a non-binding example, with the help of the accompanying drawings, in which:

FIGS. 1 to 4 represent schematic views of the overall dorsal support according to the invention in the step prior to tensioning;

FIGS. 5 to 8 represent schematic views of the overall dorsal support according to the invention in the step during tensioning;

FIGS. 9 and 10 show schematic views of the support worn by a patient using it in the two operating steps of, respectively, release and tensioning;

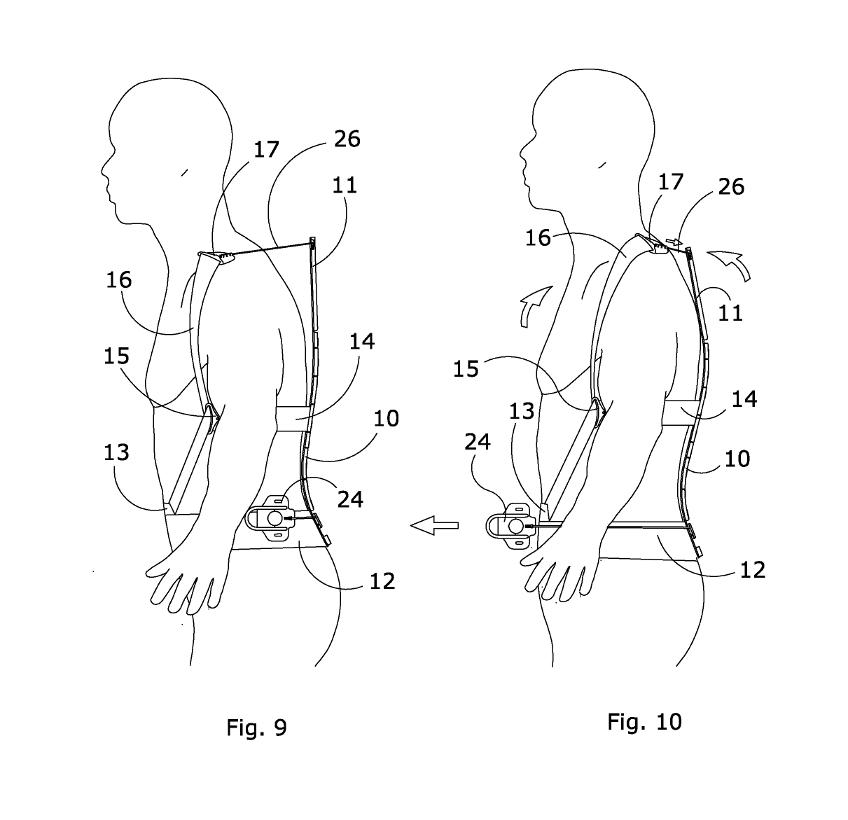

FIGS. 11 and 12 show schematic views of the upper part of the support and of the tensioning cables of the straps in, respectively, the released and tensioning steps.

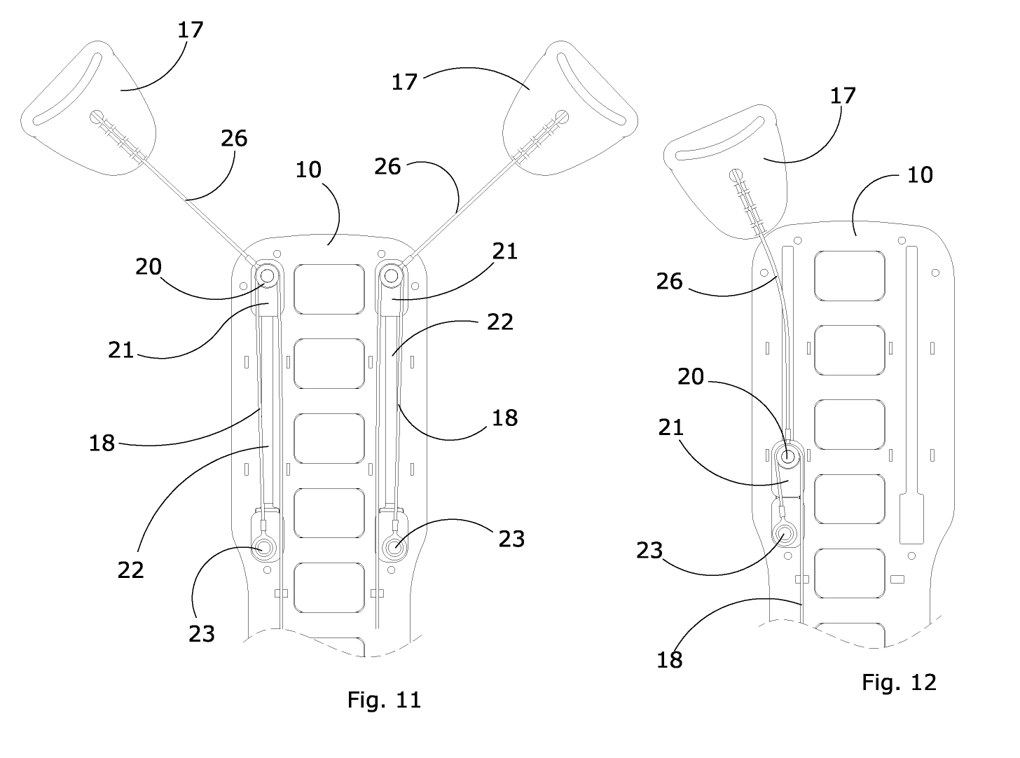

### DESCRIPTION OF ONE EMBODIMENT OF THE INVENTION

The brace tensioning system according to the invention, mainly used in the orthopedic sector for blocking and anatomically supporting the vertebral column, indicated overall with the reference number 10, therefore foresees the use of means that allow flexion of the upper part 11 of the brace 10, in order to tauten the shoulders and the upper part of the back in a backward direction, thus tending to correct the hyperkyphotic defects.

More specifically, the brace 10 consists of a semirigid slat, with elongated vertical development, shaped in accordance with the vertebral column and thus making it possible to follow and support the column, the lower part being attached to a lower strap 12 which is fixed at the front to an abdominal plate 13, wrapping around the waist, and the middle part to straps 14 which end in loops 15 positioned at the sides of the body.

Additional vertical straps 16 attached to the abdominal plate 13 pass through the loops 15 and terminate above the shoulders where loops 17 are fixed, designed to be coupled to special tie rods which tauten the orthosis.

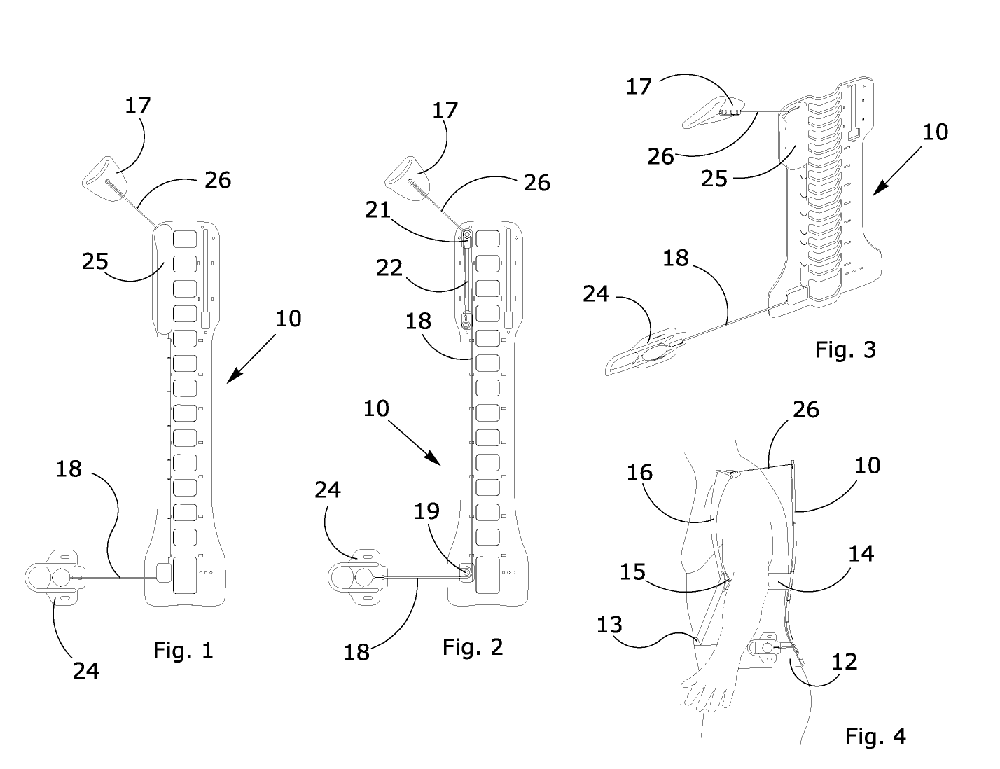

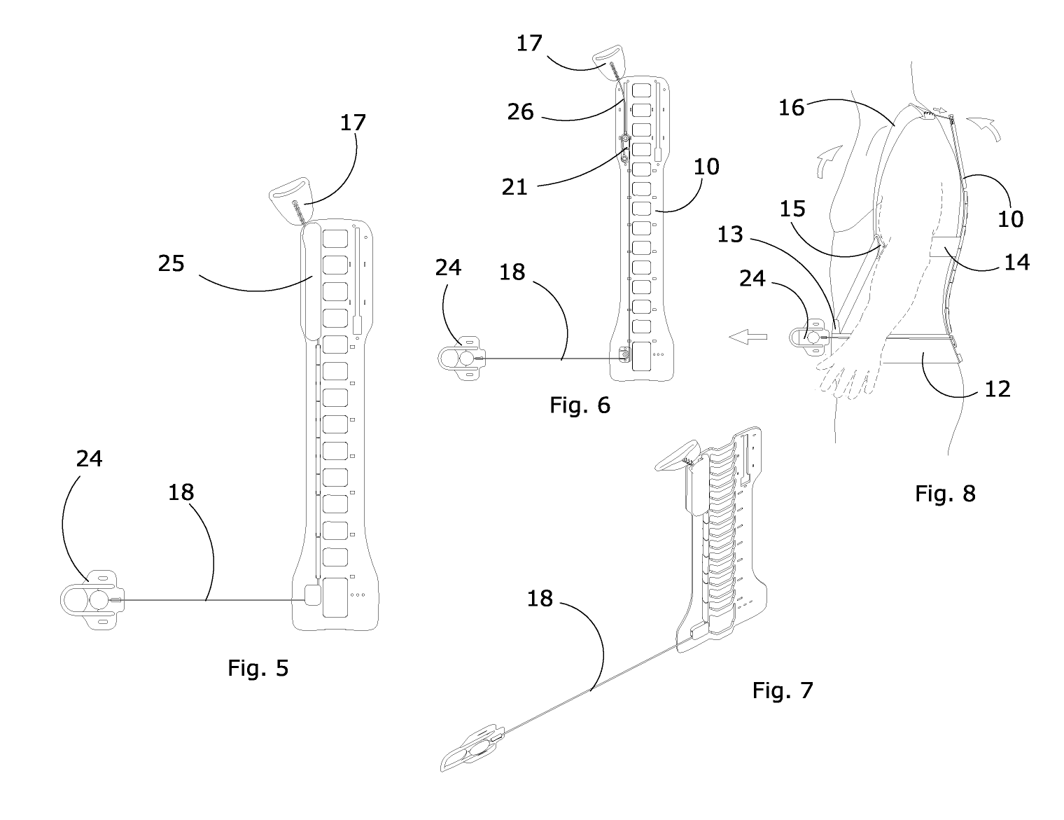

As can be seen in FIGS. 1, 2, 5 and 6, the orthosis 10 is vertically crossed by two cable tie rods 18, one on each side, which slide in relative sheaths and pass through two transmissions, that is to say a lower pulley transmission 19 and an upper pulley transmission 20 positioned on a block 21 that slides in an elongated slot 22 and locks on a button 23.

The lower end of each cable 18 terminates with a handle 24, while the slot 22, in which the block 21 slides, remains closed by a protective cover 25.

This particular tie rod system acts on the “upper strap” 16 which is no longer connected rigidly to the orthosis but to the loop 17 which is in turn connected to tie rods 18 by means of connecting cables 26, which are part of the system in question. This loop 17 is, in fact, connected, by means of a cable 26, to a block-pulley system 21-20 which is housed in a slot 22 appropriately positioned in the upper part of the orthosis.

This “block-pulley” system 21-20 is free to slide in the slot 22, controlled by the tie rod 18 which winds round the pulley 20 of the system and is locked on the button 23 positioned at the base of the slot in which the “block-pulley” system slides.

The cable 18 passes through a sheath fixed to the orthosis 10 for the entire length of the orthosis, as far as its end where it passes through the pulley transmission 19, its purpose being to divert the direction of the wire by approximately 90° so that downstream the cable terminates with the handle 24 which activates the entire mechanism.

The “block-pulley” system described above is enclosed by a cover 25, fixed with screws to the dorsal bar, which both protects and encloses the mobile and fixed parts and the cables.

From the operating point of view, the patient uses the handles 25 positioned at the height of the hips to pull forward the cables 18 attached to the handles, thanks to the geometry of the cable passage, causing the downward movement of the block 21.

Moving the block 21 downward pulls the cable 26 attached to it and to which is fixed the loop 17, through which the “armpit-shoulder” strap 16 passes; the effect therefore created is greater adherence of the upper end part of the orthosis and consequent backward tensioning of the upper part of the back starting from the patient's shoulders, tending towards greater straightening of the trunk.

The invention as described above refers to a preferred embodiment. It is nevertheless clear that the invention is susceptible to numerous variations which lie within the scope of its disclosure, in the framework of technical equivalents.

1-7. (canceled)

8. A system for tensioning straps applied to dorsal orthopedic supports, that is to say on dorsal orthoses that can be used to support and restrain the vertebral column, said orthoses consisting of a semirigid bar with elongated vertical development having a shape that corresponds to a vertebral column, making it possible to follow and support the spine, wherein the lower part is coupled to a lower strap which is fixed anteriorly to an abdominal plate that encircles the waist, and the middle part is coupled to intermediate straps that terminate at first loops positioned at the sides of the body, there also being additional vertical straps that start from the abdominal plate and pass through said first loops, characterized in that said vertical straps terminate above the shoulder and their ends are fixed to second loops, in that said second loops are coupled to respective cables which can be tensioned by a mobile slide in a sliding housing means of tie rods coupled to the orthosis and activated by the patient by means of respective handles positioned at their lower end.

9. A system for tensioning straps applied to dorsal orthopedic supports according to claim 8, characterized in that the orthosis is vertically crossed by two cable tie rods, one on each side, which slide in relative sheaths and pass through two through two transmissions, that is to say a lower pulley transmission and an upper pulley transmission positioned on a block that slides in an elongated slot and locks on a button.

10. A system for tensioning straps applied to dorsal orthopedic supports according to claim 9, characterized in that the lower end of each cable terminates with a handle, while the slot in which the block slides remains closed by a protective cover.

11. A system for tensioning straps applied to dorsal orthopedic supports according to claim 10, characterized in that said tie rods act on the upper strap, the loop of which is not rigidly connected to the orthosis, but to the tie rods by means of connecting cables, said loop being connected, by means of the cable, to the block-pulley system housed in the slot appropriately positioned in the upper part of the orthosis.

12. A system for tensioning straps applied to dorsal orthopedic supports according claim 11, characterized in that said block-pulley system is free to slide in the slot, controlled by the tie rod which winds round the pulley and is locked on the button positioned at the base of the slot in which the block-pulley system slides.

13. A system for tensioning straps applied to dorsal orthopedic supports according to claim 12, characterized in that the cable passes through a sheath fixed to the orthosis for the entire length of the orthosis, as far as its end where it passes through the pulley transmission, its purpose being to divert the direction of the wire by approximately 90° so as to be able to use the hands to operate the handle which activates the entire mechanism.

14. A system for tensioning straps applied to dorsal orthopedic supports according to claim 13, characterized in that the block-pulley system is enclosed by a cover, fixed with screws to the dorsal bar, which both protects and encloses the mobile and fixed parts and the cables.

15. A system for tensioning straps applied to dorsal orthopedic supports according to claim 8, characterized in that the lower end of each cable terminates with a handle, while the slot in which the block slides remains closed by a protective cover.

16. A system for tensioning straps applied to dorsal orthopedic supports according to claim 15, characterized in that said tie rods act on the upper strap, the loop of which is not rigidly connected to the orthosis, but to the tie rods by means of connecting cables, said loop being connected, by means of the cable, to the block-pulley system housed in the slot appropriately positioned in the upper part of the orthosis.

17. A system for tensioning straps applied to dorsal orthopedic supports according claim 16, characterized in that said block-pulley system is free to slide in the slot, controlled by the tie rod which winds round the pulley and is locked on the button positioned at the base of the slot in which the block-pulley system slides.

18. A system for tensioning straps applied to dorsal orthopedic supports according to claim 17, characterized in that the cable passes through a sheath fixed to the orthosis for the entire length of the orthosis, as far as its end where it passes through the pulley transmission, its purpose being to divert the direction of the wire by approximately 90° so as to be able to use the hands to operate the handle which activates the entire mechanism.

19. A system for tensioning straps applied to dorsal orthopedic supports according to claim 18, characterized in that the block-pulley system is enclosed by a cover, fixed with screws to the dorsal bar, which both protects and encloses the mobile and fixed parts and the cables.

20. A system for tensioning straps applied to dorsal orthopedic supports according to claim 8, characterized in that said tie rods act on the upper strap, the loop of which is not rigidly connected to the orthosis, but to the tie rods by means of connecting cables, said loop being connected, by means of the cable, to the block-pulley system housed in the slot appropriately positioned in the upper part of the orthosis.

21. A system for tensioning straps applied to dorsal orthopedic supports according claim 8, characterized in that said block-pulley system is free to slide in the slot, controlled by the tie rod which winds round the pulley and is locked on the button positioned at the base of the slot in which the block-pulley system slides.

22. A system for tensioning straps applied to dorsal orthopedic supports according to claim 8, characterized in that the cable passes through a sheath fixed to the orthosis for the entire length of the orthosis, as far as its end where it passes through the pulley transmission, its purpose being to divert the direction of the wire by approximately 90° so as to be able to use the hands to operate the handle which activates the entire mechanism.

23. A system for tensioning straps applied to dorsal orthopedic supports according to claim 8, characterized in that the block-pulley system is enclosed by a cover, fixed with screws to the dorsal bar, which both protects and encloses the mobile and fixed parts and the cables.

24. A system for tensioning straps applied to dorsal orthopedic supports according to claim 9, characterized in that said tie rods act on the upper strap, the loop of which is not rigidly connected to the orthosis, but to the tie rods by means of connecting cables, said loop being connected, by means of the cable, to the block-pulley system housed in the slot appropriately positioned in the upper part of the orthosis.

25. A system for tensioning straps applied to dorsal orthopedic supports according claim 9, characterized in that said block-pulley system is free to slide in the slot, controlled by the tie rod which winds round the pulley and is locked on the button positioned at the base of the slot in which the block-pulley system slides.

26. A system for tensioning straps applied to dorsal orthopedic supports according to claim 9, characterized in that the cable passes through a sheath fixed to the orthosis for the entire length of the orthosis, as far as its end where it passes through the pulley transmission, its purpose being to divert the direction of the wire by approximately 90° so as to be able to use the hands to operate the handle which activates the entire mechanism.

27. A system for tensioning straps applied to dorsal orthopedic supports according to claim 9, characterized in that the block-pulley system is enclosed by a cover, fixed with screws to the dorsal bar, which both protects and encloses the mobile and fixed parts and the cables.
